(19) United States
(12) Patent Application Publication
(10) Pub. No.: US 2020/0326330 A1
(43) Pub. Date: Oct. 15, 2020

(54) Device and method for multidimensional cell culture

(71) Applicant: Premas Biotech Pvt Ltd, Gurugram (IN)

(72) Inventors: Prabuddha Kumar Kundu, Gurugram (IN); Nupur Mehrotra Arora, Gurugram (IN); Avijit Das, Gurugram (IN); Saumyabrata Mazumder, Baguiati (IN); Ambica Baru, Tehsil- Sarkaghat (IN)

(21) Appl. No.: 16/775,461

(22) Filed: Jan. 29, 2020

Related U.S. Application Data
(63) Continuation of application No. PCT/IN2020/050011, filed on Jan. 4, 2020.
(60) Provisional application No. 62/814,075, filed on Mar. 5, 2019.

(30) Foreign Application Priority Data
Jan. 4, 2019 (IN) 201911000546

(51) Int. Cl. G01N 33/50; C12M 3/00; C12M 3/06; C12N 5/071; C12N 5/00

(52) U.S. Cl. CPC G01N 33/5088; C12M 21/08; C12M 23/16; C12N 2513/00; C12N 5/0062; G01N 33/5011; G01N 2500/10; C12N 5/0602

(57) ABSTRACT

The present invention discloses a device and method for multidimensional cell culture, a more particularly three-dimension (3D) and four-dimension (4D) device and method. The device and method of the present invention comprises growing cells as spheroids/tissueoids on non-woven fabric scaffold to create 3D tissue-like structures. The fourth dimension is provided by ability of the system to generate the 3D tissueoids in a much less time span and their ability to grow for extended period of time, even for greater than one year. The present invention also provides methods of use for analysis of cell-drug sensitivity of the device. Further, the invention provides a device for growth and drug sensitivity characterization of cells.

### FIELD OF THE INVENTION

The present invention lies in the field of molecular cell biology and provides cell culture devices and methods for multidimensional cellular analyses, more particularly three-dimensional (3D) and four-dimension (4D) based device and methods. Methods of making such devices are also provided. The cell culture methods and devices are useful in drug discovery and development, clinical trials, therapy decisions and a focused Patient Genome/Cancer Genome Treatment-Outcome data.

### BACKGROUND

Human physiology, pathology and tissue based analyses have until recently been done on two-dimensional (2D) cell culture methodology, which has played a pivotal role in furthering various developments in the research areas of developmental biology, tissue morphogenesis, disease mechanisms, drug discovery, tissue engineering, regenerative medicine and organ printing. There have been significant discoveries made and utilized based on this methodology and that have benefited the global population. However, as the research capabilities are undergoing a tremendous outlook change and paradigm shift, a multitude of gaps and insufficiencies associated with 2D cultures become apparent, especially with respect to the inability of 2D cultures to emulate in vivo conditions and provide physiological relevance. In the field of cancer diagnosis and medicine particularly, the gaps presented by differences between the in vivo and in vitro scenarios are a well-recognized challenge. The 2D cell-based assays have shortcomings such as design flaws, 3D spatial issues, difficult accessibility, and generally do not represent the effective 3D in vivo milieu. While rapid strides have been taken in the last few years to bridge this gap and genomic tools have been tried, they have not been fully efficient to address the complexities that exist in the patient or animal model. The gap between data generated in 2D cell based/functional assays are often 2-20-fold in dosage strengths of drugs dose titration studies that are relied upon as a rapid cut off for candidate molecules. Thus, they do not represent a real-world determination of the potential and efficacy of molecules, even further, as the animal models have their own flaws and dynamics.

The scientists in last few years have endeavoured to create in vitro or artificially, an environment for the cells in which these cells are able to grow and interact with their surroundings in three dimensions (3D). Three-dimensional (3D) cell culture is now attaining the status of new norm in the cell culture space in biomedical research field. According to current practices, 3D cultures are grown in cell culture bioreactors or miniaturized plate-based systems/capsules in which the cells can grow into spheroids, or 3D cell colonies [Goodman et al, Microsc. Microanal. 22 (Suppl 3), 2016]. Cell culturing of mammalian and human cells in 3D to create tissue like organs is revolutionizing analysis in cell culture techniques and has found application in different fields with a promising future growth.

The key to successfully growing a homotypic or a heterotypic 3D tissue culture model is to mimic the physiologic, histologic, and functional properties of the respective tissues. The homotypic system includes pure cell lines and the heterotypic system includes, for example, biopsy samples of real tumor which contain cells of mixed lineages. The 3D cell culturing methods and further development of various applications have made significant inroads into various medical, pharmaceutical and biotechnology-based applications. Numerous studies are ongoing in the field of cancer, stem cell research, drug discovery, and regenerative medicine, to name a few [Report ID: GVR-1-68038-091-0, Published Date: June 2018]. Hospitals, pharmaceutical companies, research institutes and laboratories are adopting 3D cell culture methodology and its derivations to obtain better outputs and the adoption rate is posed to increase rapidly in the next decade. The establishment of 3D cell culturing methods has been based on use of either the scaffold-based platforms, scaffold-free platforms, gels, bioreactors and/or microchips. Various scaffold-based platforms have been described in literature which are macro-porous, micro-porous, nano-porous, or solid scaffolds. However, these systems are not entirely efficient in terms of being tedious to produce and use, and are excessively time consuming, unstable over long periods, low throughput, may have biocompatibility issues with tissue samples, may pose sample retrieval challenges, etc. (Archana Swami et al., 3D Tumor Models: History, Advances and Future Perspectives; Future Oncology, May 2014).

The present invention addresses the existing challenges in the prior art to achieve a satisfying functional outcome so as to provide a multidimensional system and method that closely simulates the inner micro- and macro-scale features of the engineered tissue/s.

### SUMMARY

The present invention provides a high throughput device and method for multidimensional cell culture, more particularly three-dimensional (3D) and four-dimensional (4D) devices and methods. The device and method of the present invention comprise growing cells as spheroids and/or tissueoids on a non-woven fabric base matrix system to create 3D tissue like structures. The present invention also provides methods and devices for analysis of drug sensitivity of cells. Further, the invention provides a device for characterizing and analysing features of growth and drug sensitivity of cells, characterization of a variety of cell strains and biopsy samples.

The devices and methods of the present invention address the challenges faced in applying the existing 2D/3D systems and present a wide range of industrial applications particularly in cancer drug development, clinical trials, regenerative medicine, and personalized medicine assays, among others.

An aspect of the invention provides a device for growth of cells comprising a plurality of sterile culture chambers, each chamber containing a sterile non-woven fabric base matrix system for receiving and supporting an inoculum selected from the group of: a spheroid from a hanging drop culture, a volume of a cell culture, and a primary culture of a biopsy or an explant, each chamber having a bottom and sides for holding culture medium, the base matrix system and cells, for growth of the cells in three dimensions (3D).

In general embodiments of the device, the fabric of the base matrix system includes a non-woven matrix of polymer or copolymer fibers consisting of at least one selected from the group of: polyethylene terephthalate (PET), polypropylene (PP), polystyrene (PS), polyamide (PA), polyethylene (PE), PBT (Polybutylene terephthalate), glass fiber, acrylic resin, and cotton. Typically, the fabric of the base matrix system has a density of approximately 10-50 gm/m² and a thickness of at least about 0.05 mm and less than about 5 mm.

In general embodiments, the cells are mammalian in origin, primarily human cells, but growth of cells in the device is visualized to be possible for cells of other eukaryotic organisms including avian, reptilian, and eukaryotic micro-organisms such as yeasts. In additional embodiments, the device is employed using cells that in origin are selected from a plant, a fungal species, and a bacterial species.

In another aspect of this invention, the device helps generate a tissueoid from cells of different origin in comparatively less time for performing further screening studies, wherein the tissueoids can be visually seen in less than 72 hours or less than 48 hours or even less than 24 hours.

In another aspect of this invention, the device helps create an extracellular and intracellular architecture of the tissueoid that contains at least one component of an extracellular matrix, such that the extracellular matrix includes production and further proliferation of collagen or vascular tubules and intracellular matrix includes at least one intracellular microscopically visible structure such as tubulin and/or actin.

Another aspect of the invention herein provides a method of making a device for three dimensional growth of cells, the method including steps of: providing samples of cells selected in origin from a biopsy of a patient, an explant from a biopsy, a cell culture in a tissue culture plate, and hanging drop cultured cell spheroids, to obtain a resulting multicellular inoculum or a plurality of multicellular inocula; transferring the inoculum or inocula to a corresponding plurality of culture vessels each containing a non-woven fabric base matrix system and growth media; and, incubating the vessels to obtain the three dimensional spheroids of cells in the device. In a particularly advantageous embodiment of the method, each sample of cells contains less than about 1,000 cells, less than about 500 cells, or even less than about 250 cells, or even less than about 25 cells. Thus a single sample such as a biopsy sample or a hanging drop culture provides a plurality of aliquots of inocula for a plurality of culture vessels. The cells in the device have been demonstrated to remain viable and retain functionality for at least about 30 days, or at least about 60 days, or at least about 90 days or at least about 250 days or at least 380 days and even substantially longer.

Another aspect of the invention herein provides a method of use for analysis of cell drug response or sensitivity of a device for three dimensional growth of cell spheroids on a non-woven fabric support base matrix system, the method including steps of: contacting at least one test chamber of spheroids with at least one concentration of a drug and comparing growth and viability of the cells in the spheroid with growth in a control chamber absent the drug but otherwise identical, such that the spheroids are cultured from a patient or from a disease cell line or from a disease model animal. In a particular embodiment, at least one concentration is a plurality of concentrations of the drug in a corresponding plurality of test chambers; and/or, the drug is a plurality of drugs in a plurality of test chambers. Generally, the test chamber and the control chamber contain spheroids/tissueoids cultured from biopsy tissue from the patient of a tumor or a cultured cell line. An additional control chamber contains a spheroid/tissueoid that contains non-tumor normal cells from the patient. In a specific embodiment for a patient having a tumor, the drug is an anti-cancer chemical agent or an anti-cancer antibody or binding protein. For the patient having the tumor, an embodiment of the method includes at least one test chamber that contains a combination of two or more drugs. In alternative or additional embodiments, at least one test chamber contains a drug selected from: anti-bacterial, anti-inflammatory, anti-viral, anti-helminthic, and anti-psychotic. An embodiment of the method comprises continuing growing the spheroid/tissueoid and analysing cell functions and responses for at least about 30 days, or at least about 60 days, or at least about 90 days or at least about 250 days or at least about 380 days or even over a year.

Accordingly, an aspect of the invention provides a device for growth and drug sensitivity characterization of cells from a subject with cancer, the device comprising a plurality of sterile culture chambers, each chamber containing a sterile non-woven polyethylene terephthalate (PET) fabric base matrix system for receiving and supporting an inoculum of subject cells selected from the group of: a spheroid from a hanging drop culture, a volume of a cell culture, and a primary culture of a biopsy, and a test plurality of cultures originates from cancerous tissue from the subject, and a control culture or biopsy originates from normal tissue from the subject, each chamber having a bottom and sides for holding culture medium, the base matrix system, and cells, for characterization of growth and viability of the cells in three dimensions (3D) under a set of variable medium constituents. In a particular embodiment the device further includes the cultured cells in the chambers. For example, the sterile culture chambers are wells in a multi-well culture dish, for example, a 24 well culture dish or a 96 well culture plate.

An aspect of the invention provided herein is a set of one or more tissueoid cell cultures produced by the methods herein.

Another aspect of the invention herein provides a cell culture and artificial tissue production device comprising at least one or a plurality of sterile culture chambers, each chamber containing cells and a sterile non-woven polyethylene terephthalate (PET) fabric base matrix system for receiving and supporting an inoculum of cells, in which the cells are selected from the group of: spheroids from a hanging drop culture, volumes of a cell culture, and primary cultures of a biopsy, each chamber having a bottom and sides for holding culture media, the base matrix systems, and the cells, and each chamber having a port for addition of fresh culture medium and a drain for depletion of spent medium. In general, the cell origin is avian or mammalian. For example, the cell origin is muscle, epithelial or other tissue.

### BRIEF DESCRIPTION OF THE DRAWINGS

FIG. 1A-FIG. 1C are a set of photographs of microscopic images of three-dimensional organization of non-woven fabric as a base matrix system either empty (FIG. 1A) or in presence of MCF-7 (breast cancer cell line) tissueoids (FIG. 1B and FIG. 1C) demonstrating system provided herein to form tissue like structures. The base matrix system is a fabric mat which is made with spun-bond technology. The images (FIG. 1B & FIG. 1C) illustrate growth of tissueoids on the top of the AXTEX-4D base matrix system in a 3D fashion. In order to make a 3D culture, spheroids were formed using hanging drop method followed by growth on base matrix system resulting in tissueoid formation, using the methods and systems provided herein.

FIG. 1A shows the photograph of AXTEX-4D base matrix system in its original form without any cells or tissueoids grown on AXTEX-4D, observed by scanning electron microscope (SEM). (Magnification 100×)

FIG. 1B shows tissueoid derived from breast cancer cell line MCF-7 grown on AXTEX-4D base matrix system, observed by compound microscopy. (Magnification 40×)

FIG. 1C shows image of breast cancer tissueoid derived from MCF-7 cell line grown on AXTEX-4D base matrix system, observed by SEM. (Magnification 350×)

FIG. 2 is a set of photographs observed by phase contrast microscopy, in which HT-29 cell line grown on the base matrix system to form the 3D tissueoids. Growth of tissueoids is observed on base matrix system with different densities (19 g/m², 20 g/m², 30 g/m², 35 g/m²) of fabric mat

FIG. 3A-FIG. 3B are set of photographs of spheroids and or tissueoids showing interactions of cells with each other, resulting in cell growth in three dimension on AXTEX-4D system.

FIG. 3A shows images of spheroids (upper panel) derived from MCF-7 and HUVEC cell lines or spheroids cultured on AXTEX-4D base matrix to obtain tissueoids (lower panel). Photographs are observed by phase contrast microscopy. (Magnification 10×).

FIG. 3B is a set of images, showing structural and spatio-temporal organization of tissueoid growth on the AXTEX-4D system. Cell-cell connectivity and organization with extracellular matrix were observed, including 3D tissue like organization, cell-cell connection and interaction of organization of biopsy specimen taken from colon cancer, directly grown on the base matrix system and grown as tissueoid on AXTEX-4D system. Photographs were taken using SEM. (Magnification 1500×, 7000×).

FIG. 4A-4D shows tissueoids of transformed cell lines HEK-293 and CHO-Kl observed by scanning electron microscopy.

FIG. 4A-FIG. 4B shows the three-dimensional organization of human embryonic kidney cell line (HEK-293) tissueoids grown on AXTEX-4D base matrix system (Magnification 1000×, 1500×)

FIG. 4C-FIG. 4D shows the three-dimensional organization of Chinese hamster ovary cell line (CHO-K1) tissueoids grown on AXTEX-4D base matrix system (Magnification 1000×, 1500×).

FIG. 5 shows scanning electron microscopy images of biopsy explant taken from lung cancer, and grown as such on the base matrix system. The cells from biopsy were cultured on AXTEX-4D base matrix and growth resulted in tissueoid production without any prior treatment given to the cells. (Magnification 1000×, 1500×)

FIG. 6 shows confocal microscopy images of tissueoid on AXTEX-4D system taking PC3 cell line as an example, stained with calcein AM showing proliferation/growth as well as viability of tissueoids observed on different days i.e. 3, 25, 108 and 250 days. To determine cell viability, calcein AM cell-permeant dye was used.

FIG. 7A-FIG. 7H are a set of photographs of microscopic images of tissueoids stained with fluorescent anti-collagen type-I antibody and DAPI.

FIG. 7A-FIG. 7F show the formation of extracellular matrix in 2D monolayer and 3D tissueoids using MCF-7 cell line as an example on AXTEX-4D system. Staining was performed with anti-collagen type-I antibody (green) in 1:50 dilution, DAPI (blue—nuclei stain) on Day 7 and observed by fluorescence microscopy. The ECM formation in the tissueoids formed on AXTEX-4D system was observed to be more contiguous as seen in image (FIG. 7A-FIG. 7F) compared to that of cells cultured in 2D monolayer. (Magnification 10×). In 2D culture, the figures represent FIG. 7A—nuclei staining with Hoechst, FIG. 7B—collagen staining with anti-collagen antibody and FIG. 7C—is the merged image. In tissueoids analysis, the figures represent FIG. 7D—nuclei staining, FIG. 7E—collagen staining, FIG. 7F—merged image.

FIG. 7G-FIG. 7H are the images of entire mass of tissueoids generated from MCF-7 cell line grown on AXTEX-4D system Staining was performed with anti-collagen type-I antibody (green) in 1:50 dilution, DAPI (blue-nuclei stain) on Day 7 and observed by confocal microscopy. (Magnification 10×)

FIG. 8 compares the formation of intracellular matrix in 2D monolayer and 3D tissueoids using MCF-7 cell line as an example on AXTEX-4D system. Staining was performed with anti-phalloidin antibody (Red) in 1:1000 dilution, DAPI (blue-nuclei stain) showing that tissueoids grown on base matrix system contained cytoskeletal components. The cytoskeletal organization in the tissueoids was observed to be more contiguous as seen in image above (FIG. 8) compared to that of cells cultured in 2D monolayer. (Magnification 10×)

FIG. 9A-FIG. 9F show tissueoids generated from various cell numbers of MCF-7 cells, with a range between ˜250 to ˜25 cells using phase contrast microscope. Tissueoids were grown on AXTEX-4D system. FIG. 9A, 9B, 9C, 9D, 9E are images from phase contrast microscopy study (10× magnification) and FIG. 9F is an image using scanning electron microscope for ˜25 cells grown on AXTEX-4D system. (Magnification 1500×). Inocula in a range of cell numbers from 5000 cells and less were evaluated and the data shown here depict successful growth on the AXTEX-4D system of about 250 cells to as less as 25 cells.

FIG. 10A-FIG. 10C is a set of photographs showing morphological characteristics of MCF-7 cells either grown as 2D monolayer culture or as tissueoids grown on AXTEX-4D system.

FIG. 10A-FIG. 10B is a set of photographs that show morphology of MCF-7 cells treated with or without doxorubicin after 3 days grown in 2D culture. Dose-dependent growth inhibition was observed in treated group compared to that of cells cultured in presence of DMSO (vehicle control). Vacuoles were observed even at 1 μM doxorubicin in 2D culture indicating the sensitivity of cells towards doxorubicin treatment which eventually led to cell death. A portion of the insets of the photographs from FIG. 10A are shown as a magnified view in the photographs in 10B.

FIG. 10C is a set of photographs that show sensitivity of MCF-7 tissueoids against doxorubicin after 3 days of incubation. Growth of tissueoids generated from MCF-7 cell line was not inhibited at 2.5 μM concentration of doxorubicin and was comparable to the growth observed in the tissueoid growing without the drug in presence of vehicle control (DMSO). Partial growth inhibition of tissueoid was observed at 5 μM concentration. At higher concentration (5 tissueoids did not disintegrate from the AXTEX-4D base matrix system but remained attached to it, though shrinking of the tissueoid was visible.

FIG. 11A-FIG. 11B are set of bar graphs that describe the sensitivity of MCF-7 cells either grown in a 96 well plate as a monolayer or tissueoid on AXTEX-4D system towards doxorubicin at indicated concentrations in presence or absence of bevacizumab antibody.

FIG. 11A shows drug sensitivity analysis using three different concentrations of doxorubicin in both 2D monolayer culture and 3D tissueoid system for 48 hrs. The viability of the cells in each of monolayer culture (2D) and in tissueoid (3D) was evaluated by analysing viability using prestoblue. The data is expressed in relative fluorescence unit (RFU) and normalized with the vehicle control as 100% viability. Resistance of the drug activity was observed in tissueoids grown on AXTEX-4D system even at 1 μM of doxorubicin concentration (˜80% viability). At the same concentration (1 μM of doxorubicin), cells cultured in 2D monolayer showed 35% viability.

FIG. 11B shows the combinatorial effect of doxorubicin and bevacizumab on VEGF-165 induced MCF-7 cell proliferation grown as tissueoids. MCF-7 tissueoids were cultured in wells of a 96 well plate. Cells were serum starved for about 5 hrs and subsequently treated with 100 ng/ml of VEGF-165 either alone or in combination with 1 μM doxorubicin and 25 μg/ml of bevacizumab for 6 days. Viability of the cells was analysed using prestoblue. Tissueoids grown on AXTEX-4D system showed greater efficacy of combination effect of both drugs (about 57%) as compared to monotherapy (as shown in FIG. 11A) to prevent cell growth.

FIG. 12 represents phase contrast image of tissueoids of HT-29 before (FIG. 12A) and after treatment (FIG. 12B) with cytokine TNF-α (20 ng/ml) in combination with IFN-γ (0.5 ng/ml) for 16-18 hrs. Intact tissueoids became fragmented and dislodged from AXTEX-4D base matrix system after treatment with cytokine demonstrating impact of cytotoxicity.

FIG. 13 is a set of photographs that shows the duration in length of time (in days as indicated in each panel) during which the tissueoids remained in culture. Using phase contrast microscopy, the longevity of tissueoids derived from HepG2 and PC3 cells was observed. Different fields on different days were captured and increased number of cells with increasing density was observed. PC3 tissueoids are viable in culture as of the date of filing of the present application. (Day 364). Viability of Hep-G2 tissueoids was observed until day 82. Viability of PC3 tissueoids was observed until day 364.

FIG. 14 is a set of photographs that shows mono, co and tri culture of three cell lines by adding cell suspension of transformed fibroblast cell line (NIH-3T3), endothelial cells (HUVEC) and breast cancer cell line (MCF-7) and grown as 2D monolayer culture or as tissueoids on base matrix system AXTEX-4D. Co-culture of each of the combinations were analysed by taking either breast cancer cell line (MCF-7) and endothelial cells (HUVEC) or endothelial cells (HUVEC) and fibroblast (NIH-3T3) in 1:1 ratio respectively. For tri-culture NIH-3T3, HUVEC and MCF-7 cells lines were-added in 2:1:1 ratio. The spheroids were formed for all combinations and cultured in 2D monolayer as well as on the AXTEX-4D base matrix system. Attachment of spheroid was observed within 24 hrs on the AXTEX-4D base matrix system and all combinations were observed to have grown further, as tissueoids.

FIG. 14 (upper panel) shows monolayer culture of spheroids made of HUVEC, HUVEC: MCF-7, HUVEC: 3T3 and HUVEC: MCF-7:3T3 grown on 2D format.

FIG. 14 (lower panel) shows the tissueoids of HUVEC, HUVEC: MCF-7, HUVEC:3T3 and HUVEC: MCF-7:3T3 grown on AXTEX-4D base matrix system.

FIG. 15 is a set of photographs that show tissueoids of HEK-293, NIH-3T3 and PC3 grown on AXTEX-4D system. FIG. 15 shows minimum time taken for tissueoids to have adhered and have initiated growth on the AXTEX-4D system. Tissueoids of HEK-293 and NIH-3T3 cell lines took less than24 hrs for attachment and proliferation of cells on AXTEX-4D system whereas tissueoids of PC3 took approximately 48 hrs to adhere and further grow on AXTEX-4D system.

FIG. 16A-FIG. 16B is a set of photographs that shows application of AXTEX-4D system as cell factory. Adherent CHO-DG44 stable cell line expressing tocilizumab is growing as a tissueoid on AXTEX-4D base matrix system and secretion of monoclonal antibody Tocilizumab is observed in the culture supernatant, as analysed by SDS PAGE.

FIG. 16A shows growth of tocilizumab expressing CHO-DG44 cells as tissueoids on AXTEX-4D.

FIG. 16B shows expression analysis of tocilizumab by non-reducing SDS-PAGE. Briefly, culture supernatant was taken out from different day's culture from cells growing on petri-plate as 2D culture and tissueoids grown on AXTEX-4D system. Tissueoid grown on AXTEX-4D base matrix system, allowed increased number of cells in a more compact space, with increased longevity (6 days in monolayer and 26 days as tissueoids when the samples were taken out for analysis) and better productivity. SDS PAGE analysis showing expression of monoclonal antibody (Tocilizumab) in adherent CHODG44 cell line in 2D as well as 3D format; each lane of 10% SDS-PAGE was loaded with a different sample; Lane 1: Prestained protein marker, Lane 2: Positive control (1 μg), Lane 3: Day 6 sample of 2D culture supernatant, Lane 4: Day 6 sample of tissueoids supernatant, Lane 5: Day 12 sample of tissueoids supernatant, Lane 6: Day 18 sample of tissueoids supernatant, Lane 7: Day 26 sample of tissueoids supernatant . Equal number of cells were seeded on 2D as well as 3D format. After 6 days 2D culture was terminated due to confluency of culture of tissueoids was sustained till day 26. SDS-PAGE revealed that tocilizumab antibody expression was observed on day 6 in 2D monolayer culture, whereas no expression was observed in tissueoids grown on AXTEX-4D on day 6. However, the expression of tocilizumab in 3D culture was observed to have increased as a function of time of incubation in days from day 12 to day 26.

FIG. 17A-FIG. 17C are set of photographs that show growth of endothelial cells HUVEC as tissueoids on AXTEX-4D base matrix system in presence (FIG. 17B) and absence of VEGF-165 treatment (FIG. 17A) for 72 hrs and observed by phase contrast microscopy. FIG. 17A shows attachment of spheroid to the base matrix system with minimal proliferation. FIG. 17B shows proliferation of cells along with tube like structure formation demonstrating that angiogenesis was observed in AXTEX-4D system. FIG. 17. C (magnified view of tissueoid with VEGF treatment) is the magnified view depicting closer look at the tube-like structure.

FIG. 18 is a drawing of an embodiment of the invention which is a device with the 3D and 4D elements.

FIG. 19 is a drawing that shows various uses of the devices provided herein and relative advantages.

### DETAILED DESCRIPTION OF THE EMBODIMENTS

The following description with reference to the accompanying drawings is provided to assist in a comprehensive understanding of exemplary embodiments of the invention. It includes various specific details to assist in that understanding but these are to be regarded as merely exemplary.

While the invention is susceptible to various modifications and alternative forms, specific embodiments thereof have been described in detail below. It should be understood, however that it is not intended to limit the invention to the particular forms disclosed, but on the contrary, the invention is to cover all modifications, equivalents, and alternatives falling within the spirit and the scope of the invention. In addition, descriptions of well-known functions and constructions are omitted for clarity and conciseness.

The terms and words used in the following description and claims are not limited to the bibliographical meanings, but, are merely used by the inventors to enable a clear and consistent understanding of the invention. Accordingly, it should be apparent to those skilled in the art that the following description and embodiments of the present invention are provided for illustration purpose only and not for the purpose of limiting the invention as defined by the appended claims and their equivalents.

Features that are described and/or illustrated with respect to one embodiment may be used in the same way or in a similar way in one or more other embodiments and/or in combination with or instead of the features of the other embodiments.

It should be emphasized that the term “comprises/comprising” when used in this specification is taken to specify the presence of stated features, integers, steps or components but does not preclude the presence or addition of one or more other features, integers, steps, components or groups thereof.

Accordingly, the present invention relates to cell culture systems for multidimensional, particularly, 3D/4D systems for cellular and molecular studies. A device encompassing the novel 3D/4D tissue culture models is also provided. Further, the present invention provides methods of preparing the said multidimensional cell culture systems.

An objective of the present study is to provide cell culture systems for multidimensional tissue model analyses. More particularly, 3D/4D tissue culture and tissueoid generation system for cellular and molecular analyse and further applications of it.

Yet another objective of the present invention is to provide method of preparing the aforesaid systems and culture devices.

Yet another objective of the present invention is to provide a high throughput device for growth of cells in which it is containing a plurality of sterile culture chambers, each chamber containing a sterile non-woven fabric base matrix for receiving and supporting an inoculum selected from the group of a spheroid from a hanging drop culture, or direct suspension of cells derived from tissue, a volume of a cell culture derived from cell lines, and a primary culture of a biopsy or an explant, each chamber having a bottom and sides for holding culture medium, the base matrix and cells, for growth of the cells in three dimensions (3D). The devices provided herein contain a non-woven mat of polymer fibers consisting of at least one selected from the group of: PET, PP, PBT, glass fiber, and cotton.

In another embodiment, devices are provided, the fabric of the base matrix has a density of in range of approximately 10 gm/m² and 50 gm/m², for example 19-25 gm/m² and a thickness of at least about 0.05 mm and less than about 5 mm, for example 0.12 mm. The thickness of the fibers is 0.5-10 dtex, for example 2.5-3.0 dtex and the porosity in range of 20-80 micron.

In another embodiment, the device of the present invention is used to grow cells selected from a mammalian species. In embodiments, the mammalian cells are human, such as established cell lines or fresh biopsy samples from a patient, or are other mammalian cells such as Chinese Hamster ovary derived cells (CHO and CHO derived cells).

The tissueoids that have been grown successfully using this technique include the following cancerous cell lines: MCF-7: breast cancer cell line from an adenocarcinoma; HepG2: liver carcinoma of epithelial cells; PC3: prostate cancer cell line from an adenocarcinoma; and A375: skin melanoma which is an epithelial cell line.

The following non-malignant cell lines have been successfully grown: as shown in examples herein CHO cells (Chinese hamster ovary); HEK-293 (human embryonic kidney cells); and NIH-3T3 (Fibroblasts).

Primary tissues that have also been successfully used as a source of cells include: breast cancer tissue from a tumor; colon cancer from a tumor; gastric cancer from a tumor; lung cancer from a tumor and; thyroid cancer from a tumor.

In general embodiments of the device, tissueoids grown by the methods herein produce extracellular architecture Le, collagen. Tissueoids growing in this base matrix AXTEX-4D system were observed to produce 3D like rearrangement of cytoskeletal elements by analysing expression of F-actin. Tissueoids continued to proliferate for an extensive period of time indicating favourable growth conditions provides by the device here in.

An embodiment of the invention provides a method to grow a cell sample employing as an inoculum a sample containing less than about 1,000 cells, less than about 500 cells, less than about 250 cells and even less than 25 cells. The cells are derived from stable cell lines or from living tissues such as tumor biopsies that are cultured ex vivo using the device and methods herein.

In another aspect, the invention provides a method of use for analysis of cell-drug sensitivity of a device for three dimensional growth of tissueoids cultured from cells of a patient, on a non-woven fabric support base matrix in which it is contacting at least one test chamber of tissueoids with at least one concentration of a drug, and comparing growth and viability of the cells in the tissueoids with a control chamber absent the drug but otherwise identical.

In an embodiment, at least one concentration is a plurality of concentrations of the drug in a corresponding plurality of test chambers; and/or, the drug is a plurality of drugs in a plurality of test chambers. The drug is selected from an anti-cancer chemical agent or an anti-cancer antibody or binding protein.

In another preferred embodiment, the test chamber and the control chamber contain tissueoids cultured from biopsy tissue from the patient of a tumor. In yet another embodiment, an additional control chamber contains tissueoids containing non-tumor physiologically normal cells from the patient is provided.

In another embodiment, at least one test chamber contains a drug or a combination of two or more drugs. In a further embodiment, at least one test chamber contains a drug selected from: anti-bacterial, anti-inflammatory, anti-viral, anti-helminthic, and anti-psychotic.

In another aspect of the invention, a device for growth and drug sensitivity characterization of cells from inocula with cancer is provided. The device comprises a plurality of sterile culture chambers, each chamber containing a sterile non-woven polyethylene terephthalate (PET) fabric base matrix for receiving and supporting an inoculum of cells. The inocula are selected from the group of: a spheroid from a hanging drop culture, a volume of a cell culture, and a primary culture or explant from a biopsy, such that a test plurality of cultures originates from cancerous tissue from the inocula, and a control culture or biopsy originates from normal tissue from the inocula. Each chamber has a bottom and sides for holding culture medium, the base matrix, and cells, for characterization of growth and viability of the cells in three dimensions (3D) under a set of variable medium constituents. In a further embodiment, the cultured cells are present in the chambers in a multi-well culture dish, for example, a 24 well culture dish or a 96 well culture plate.

The present invention also provides a cell culture and ex vivo tissue production device including at least one or a plurality of sterile culture chambers, each chamber containing cells and a sterile non-woven polyethylene terephthalate (PET) fabric base matrix for receiving and supporting an inoculum of cells. The cells may be selected from the group of: spheroids from a hanging drop culture, volumes of a cell culture, and primary cultures of a biopsy, each chamber having a bottom and sides for holding culture media, the base matrix, and cells, each chamber having a port for addition of fresh culture medium and a drain for depletion of spent medium.

The invention provided a device having tissueoids in which the cell origin is avian or mammalian.

In an embodiment, the cell origin may be selected from muscle, epithelial or other tissue. The present invention also provides a use of the resulting production by the device for a therapeutic artificial skin or muscle.

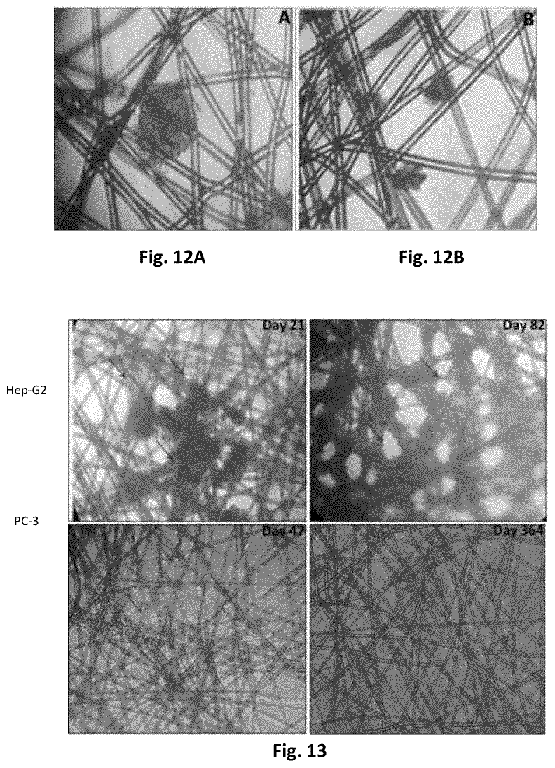

In another aspect, the cells of the tissueoids produced by the method and devices provided herein of the present invention have longer lifespans, viz., longer period of time of cell viability compared to what is reported in the prior art. A viability of up to 250 days has been observed in the present examples with different cell lines, as shown in FIG. 6 and further continued growth of cells has been shown up to 364 days, as shown in FIG. 13. It has been observed that the tissueoids remain viable with adequate form and function for a period of time such as more than 12 months. Various examples have been conducted with different cell lines and primary cells and the longevity of the tissueoids has been reproducibly seen to be substantially greater than that reported earlier.

In another aspect, the present method and device provides 3D culture assays that are initiated in less time (less than 72 hours) than reported previously. Zanoni M et al. forms spheroids using hanging drop method by using 2×10³, 4×10³, 6×10³ cells/well, however these spheroids were reported to need a period of 7 days.

TABLE 1
Scaffold free techniques for obtaining tumor spheroid models.

| | | | Equivalent | | |
|---|---|---|---|---|---|
| | | | diameter [μm] | Amount of | |
| | ◌^{∫}Time | ◌^{∫}No. Cell | (range, | spherical | Amount large |
| | required | Required | mean ± SD, | spheroids | spheroids |
| | [day] | [×10 | CV, n) | (SI ≥ 0.90) | (500 μm) |
| Magnetic | 7 | 0.5 | 200-500, | Low | Low |
| Levitation* | | | 347 ± 87, | | |
| | | | 25.1, 28 | | |
| Hanging | 7 | 0.6 | 200-500, | Low | Low |
| drop^{~} | | | 359 ± 95, | | |
| | | | 26.5, 38 | | |
| Pellet | 1 | 20 | 800-900, | High | High |
| Cultures [symbol] | | | 880 ± 21, | | |
| | | | 2.4, 20 | | |
| Rotating Wall [symbol] | 15 | 40 | 500-1100, | Low^{x} | High |
| Vessel (NASA | | | 897 ± 98, | | |
| Bioreactor) | | | 11.0, 192 | | |

[*Haisler, W. L. et al. Three-dimensional cell culturing by magnetic levitation. Nat. Protoc. 8, 1940-1949 (2013).
^{~}Kelm, J. M., Timmins, N. E., Brown, C. J., Fussenegger, M. & Nielsen, L. K. Method for generation of homogeneous multicellular tumor spheroids applicable to a wide variety of cell types. Biotechnol. Bioeng. 83, 173-180 (2003). [symbol]
Johnstone, B., Hering, T. M., Caplan, A. I., Goldberg, V. M. & Yoo, J. U. In vitro chondrogenesis of bone marrow-derived mesenchymal progenitor cells. Exp. Cell Res. 238, 265-272 (1998). [symbol]
Ingram, M. et al. Three-dimensional growth patterns of various human tumor cell lines in simulated microgravity of a NASA bioreactor. In Vitro Cell. Dev. Biol. Anim. 33, 459-466 (1997).]

In the device and the method of the present invention, the spheroids were generated in about 24 hours or less. Further, such spheroids were able to bind onto the base matrix in less than about 24 hours. In another preferred embodiment of this invention, tissueoids show growth on the base matrix in less than 24 hours.

Yet another advantage was that the device and method are capable of using different cell lines. Even the types of cells which were usually less compact in nature, such as the PC3 cell line (Prostate cancer) and HT 29 cell line (Colorectal cancer) were observed to display good binding to the base matrix. The 3D/4D device and method of the present invention were observed to grow tissueoids that in function and structure were observed to be similar to the original tissue across all cell lines tested herein.

In yet another aspect, the device and method of the present invention yield results that are comparable to genomic and proteomic profile of the primary tumor tissue and gives results that supersede studies done on monolayer cultures, results of which may be inconsistent and non reproducible.

In aspects of the present invention, the multi-dimensional physical and analytical readout properties and applications of this device/base matrix AXTEX-4D system are the following. Tissueoids were grown either as single pure culture, or as combinations of multiple types of cells; as adherent or in suspension and combinations thereof, for either support, sustenance or similar to what was observed in an in vivo network to simulate the micro-environment in-vivo of the particular organs, tumor, or interplay of the immune system against cancers or infections. Multiple combination therapy/ies using one more combinations of both chemical and biological drugs was designed, tested and evaluated. Genetic changes due to impact of administration of the drugs/combinations in varied doses/dosage forms is determined as a function of time, by accelerated studies. Physico-stimulation of the tissueoid to attach, proliferate and promote accelerated growth was tested to deliver rapid test results for drugs impact, efficacy studies, genetic mutations, etc. For example, the 3D/4D models provided in the present invention is used also to study the concentration of various drugs that can be used as effective doses for treatment regimen.

A fast turnaround time for obtaining results allows this model to yield more effective analysis and benefit the patients in their clinical outcome.

In another embodiment, the systems of the present invention are used to establish the drug combination therapy using both chemical and biological drugs. The systems are envisioned as useful to select patients for clinical trials in oncology related trials, to decide on multiple therapy regimens and multiple concentrations is studied at the same time i.e., simultaneously, to generate data from patients having a disease, and to correlate with data from the tissue growing in vitro, and the data received after impact of treatment. Thus, a focused Patient Genome/Cancer Genome Treatment-Outcome database is generated to enable effective future treatment regimens, by using the ex vivo AXTEX-4D base matrix system provided herein to screen for patients, to identify appropriate efficacious therapeutic agents for each patient.

In yet another embodiment, the invention provided methods and devices for validation of tissue like structures having the ability to support various tumor cell lines in 3D; and a fourth dimension (4D) as a function of an extended period of time, and applications thereof in drug discovery and other clinical analyses, diagnosis etc.

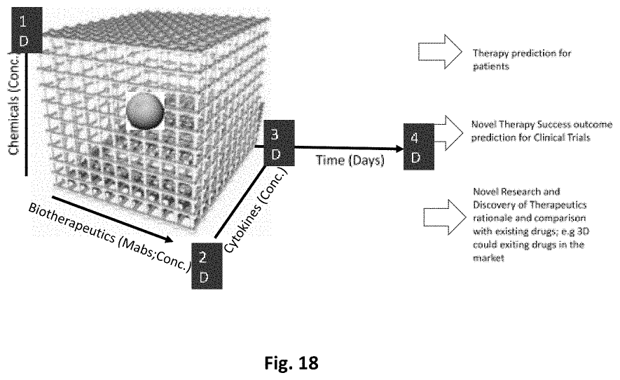

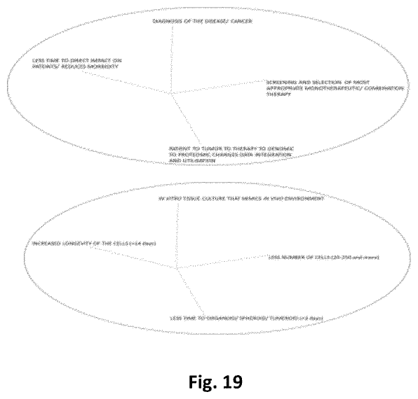

The device encompassing the 3D tissue system and its configuration is presented in FIG. 18 which demonstrates a 3D tumor base matrix system central to the cubic design. In an embodiment, the device of the present invention is provided as a 4D base matrix system which is used for studies on tissues under in situ conditions which allow efficient monitoring and evaluation of input and device output as a function of time.

The dimension of time when included in execution of cell culture in vitro, provides methods of quicker diagnosis of disease as compared to existing methodologies. Further, it also increases the window of trial for different analyses due to increased longevity of the tissueoids created by the methodology described herewith. The AXTEX-4D base matrix system envisaged in the present invention to be used for longer periods of time is pictorially depicted in FIG. 6.

In another important aspect of this invention, the device helps generate a tissueoid from cells of different origin in comparatively less time for performing further screening studies, wherein the tissueoids can be visually seen in less than 72 hours or less than 48 hours or even less than 24 hours.

An important observation during the studies and thus an important aspect is that there is evidence of tubule like structures growing and thus it can possibly be said that the present invention provides an angiogenesis model to study antiangiogenic drugs/assays and other applications

In an embodiment, the systems of the present invention are useful as a source of material in determining the proteomic and genomic profile.

In yet another embodiment, the device is used as a biotransformation reactor, for example to generate high value proteins such as antibodies during a time course extending for months.

In yet another embodiment is provided a method of determining an efficacious treatment or regimen of treating diseases like cancer.

In yet another embodiment the system of the present invention can be used as a cell factory/bioreactor, to grow large cultures and produce therapeutics/antigens/vaccine candidates etc.

Overall, the systems of the present invention are more rapidly growing, robust, viable and sustainable for a longer time, with close representation of tissue like structure and function.

### EXAMPLES

The present invention is described below in further detail with examples and comparative examples thereof, but it is noted that the present invention is by no means intended to be limited to these examples.

### Example 1: Compounds and Reagents

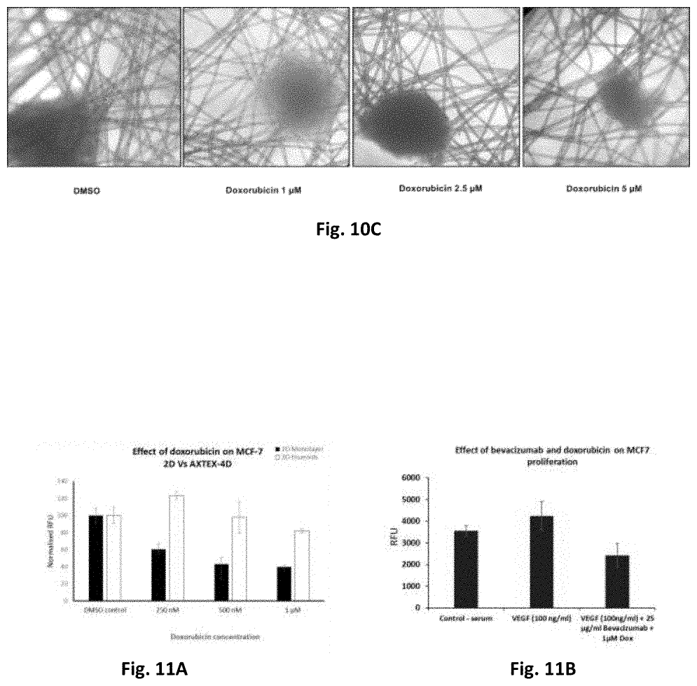

Compounds and reagents that were used for drug sensitivity analysis using the device and methods provided as a base matrix system AXTEX-4D. Doxorubicin, Cisplatin, Colchicine, Paclitaxel and DMSO were purchased from Sigma. These drugs were tested for sensitivity/resistance of cells of different cancer cell lines grown as 2D (Monolayer) and as 3D (On the AXTEX-4D base matrix system as tissueoids) formats and the data were compared. Exemplary data are shown in FIGS. 10 and 11.

### Example 2: Cell Lines and Tumor Analyses

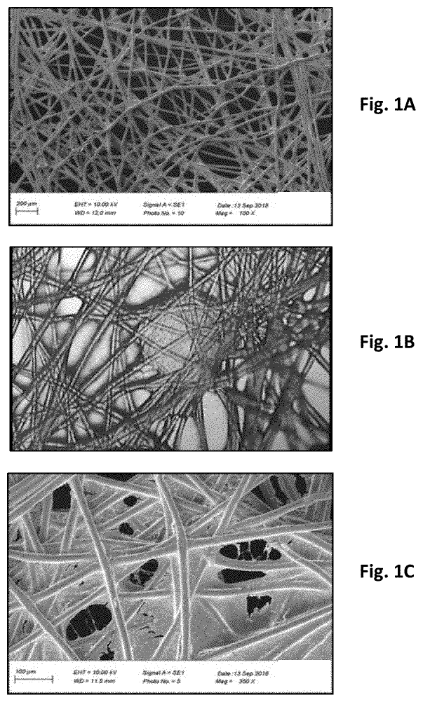

The various human cancer cell lines (such as MCF-7, HepG2, PC3, HT29) were obtained from the American Type Culture Collection (ATCC, Rockville, Md.) A375 and CHO-K1 cell lines were received from NCCS, Pune, India. HUVEC was obtained from Lonza. MCF-7 and HepG2 were cultured in EMEM (Sigma-Aldrich, St. Louis, Mo., USA). PC3 and CHOK-1 was cultured in F12K (Sigma-Aldrich, St. Louis, Mo., USA) HT-29, A-375, NIH-3T3, HEK-293 cells were cultured in DMEM (Sigma-Aldrich, St. Louis, Mo., USA). HUVEC cells were cultured in EBM-2 basal medium and EGM-2 Single Quots supplements. FIGS. 1, 2 and 3A and 4 are representative photographs showing growth of cell lines on 3D base matrix system to form tissueoids. All the adherent cell lines were cultured in presence of 10% FBS (Gibco) and supplemented with 2 mM glutamine (Sigma-Aldrich, St. Louis, Mo., USA). Cells were cultured at 37° C. humidified condition with 8% CO2 under static condition.

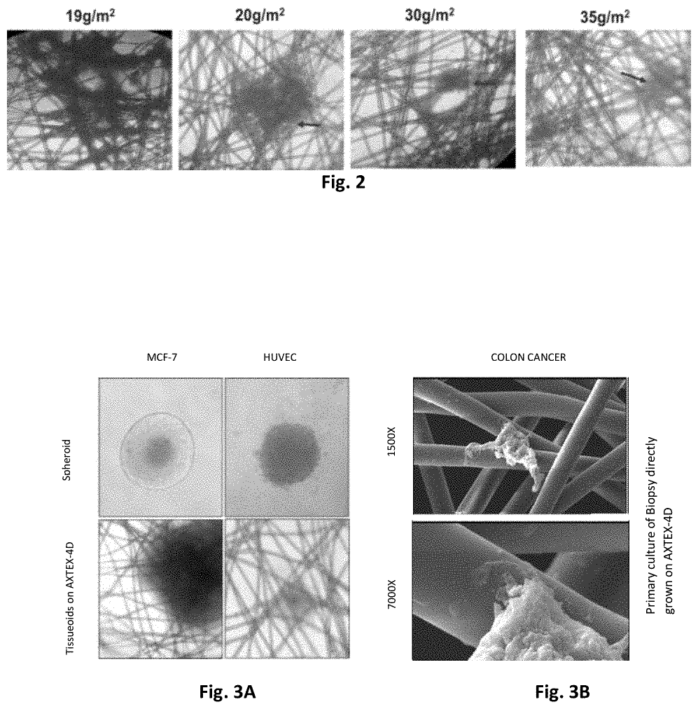

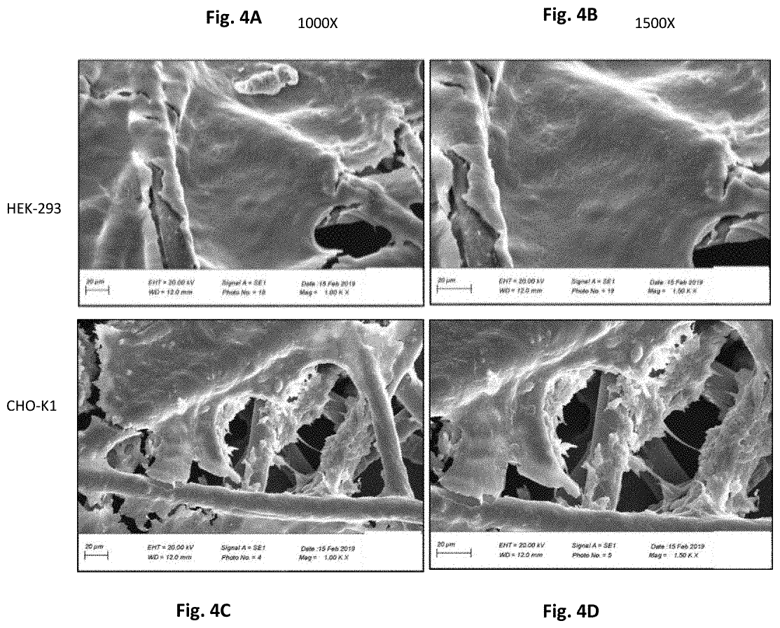

The morphological appearance of tissueoids for each of the cell lines was analysed as phase contrast and SEM images in (FIG. 1, FIG. 2. FIG. 3A, FIG. 4A-FIG. 4D).

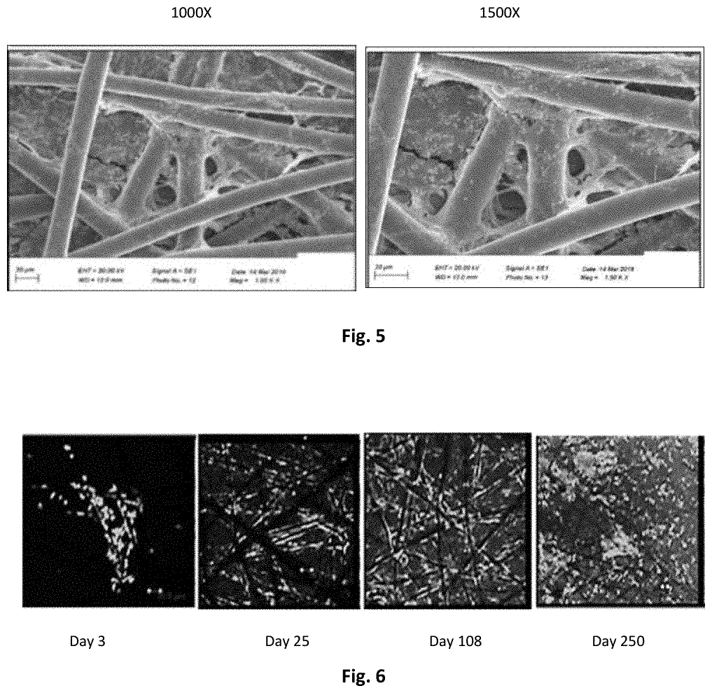

Generation of tissueoids using primary tumor biopsy: Tumor biopsy samples for each of colorectal, gastric, lung and thyroid carcinoma were collected from pathology specimens, transported for culturing, and analyzed ex vivo in cell culture lab. Tissue was rinsed with 1× PBS (without Ca⁺⁺ and Mg⁺⁺) thrice and sliced with a scalpel into smaller pieces and further processed with the plunger in order to separate and isolate the cells. Cells were cultured then in DMEM media containing 2 mM glutamine 2× antibiotic solution (Penicillin and Streptomycin, Himedia) and 20% FBS. Tumor tissue specimens were taken as suspension culture or as explant and grown on AXTEX-4D system. The growth of tumor tissue on the AXTEX-4D base matrix system is shown as an example in. FIG. 3B and FIG. 5.

Spheroids and Tissueoids formation. Unless otherwise indicated, spheroids were formed by using hanging drop method. This further resulted into formation of tissueoids. FIG. 3A (upper panel), showing development of a spheroid generated by the hanging drop method and FIG. 1, FIG. 2, FIG. 3A (lower panel), FIG. 4A-FIG. 4D is a set of representative photographs of tissueoids growing on the AXTEX-4D base matrix. The process for the spheroid formation of various cell lines is described below:

Briefly, cells were seeded at approximately 80% confluency the day before making hanging drops. After trypsinization, cells were resuspended in an appropriate volume of respective media and the process of hanging drop formation was initiated only when the viability of the cells was more than 90%. Each cell suspension was made such that 20 μl of the media contained a cell number in a range of 10³-10⁴ cells. The drop was pipetted onto the inner surface of a lid of a sterile culture dish and PBS was filled in the bottom of the dish. After 24-48 hrs, the inner lid was inverted and the drops were re-suspended in a fresh media. Spheroids were analyzed by phase contrast microscopy. Representative photograph showing development of a spheroid using the hanging drop method in FIG. 3A (upper panel) and tissueoids grown on 3D base matrix system (FIG. 1B, FIG. 1C, FIG. 2, FIG. 3A (lower panel) and FIG. 4A-FIG. 4D.

Scanning electron microscopy. 3D morphology of the cells attached to the base matrix AXTEX-4D system was evaluated by SEM analysis (EVO-18 Research, Zeiss) (FIG. 1C, FIG. 4A-FIG. 4D). Samples (fixing agents: 2.5% glutaraldehyde and 2% paraformaldehyde in PBS, pH 7.4) were fixed on the top of a stub, vacuum dried for 10 mins with 0.1 mbar pressure followed by addition of argon gas. Samples were coated with gold particles using Sputter Coater. Coated samples were then analyzed by scanning electron microscopy.

### Example 3: Preparation of the 3D Cell Culture System:

A commercially available spun-bound PET material consisting of extruding round continuous filaments (FIG. 1A) which are flat bond, was used in the examples herein. The fabric used is a non-woven mat of endless polymer fibers. The density of the fabric is approximately 19-35 gm/m² with a porosity of approximately 65 micron. In order to make a tissueoids, spheroids were prepared using hanging drop method and these were cultured for growth on the top of the base matrix (FIG. 1B, FIG. 1C and FIG. 3A (lower panel).

The attachment and growth of the tissueoids was continuously monitored as a function of time using phase contrast microscopy. It was observed that the entire process was completed in less than 24 hours or less than 48 hours or less than72 hours; including approximately 24 hrs to prepare the spheroids, 24 hrs to attach the spheroids on the base matrix system and a few hours for spheroids to proliferate and generate as tissueoids. After this, the AXTEX-4D system (cells growing on base matrix system in 3D culture) was ready to conduct screening studies and other analyses demonstrated in other examples. In this tissueoid base matrix system, spheroids of various primary cells and tissues, pathological and non-pathological, cancer cells or patient tumor biopsies, transfected and non-transfected cell lines were observed to be grown with similar morphology to tissues in vivo.

Tissueoids was also generated from tumor biopsy by taking either suspension culture and or explant. Growth of cells of an explant inoculum to form a tissueoid was observed in less than 24 hours or less than 48 hours or less than 72 hours of incubation in the 8% CO2 incubator. After this, the platform was ready to perform screening studies and other analyses.

### Example 4: Types of Materials of Construction and Thickness Parameters for Base Matrix System

Different types of spun woven fabric materials, such as PET fabric with a various density (19, 20, 30, 35) gm/m² were used as 3D base matrix system. A representative example, FIG. 2 shows efficient growth of HT-29 tissueoids each on the same fabric and having different densities (ranges from 19, 20, 30 and 35 gm/m²). Other materials that were also tested as base matrix system to grow tissueoids included: FNT best bond PP/PS/PA-40 g/m²²; FNT Cisellina PET 250 g/m²; FNT Newjet viscose 80 g/m²; FNT Polibond PP 45 g/m²; Hydroweb BicoPET/PP 150 g/m²; JM 011/120 PET/120 g/m²; Mogul Buffalo bico PET/coPET, round 80 g/m²; Mogul Buffalo bico PET/coPET, tiptrilobal 80 g/m²; Mogul Mopet PET flatbond 19 g/m²; Mogul Mopet PET flatbond 75 g/m²; Resintex Master PE, acrylic resin 220 g/m²; AS10; AS03; and ASO3A.

### Example 5: Cell-Extra Cellular Matrix Interaction

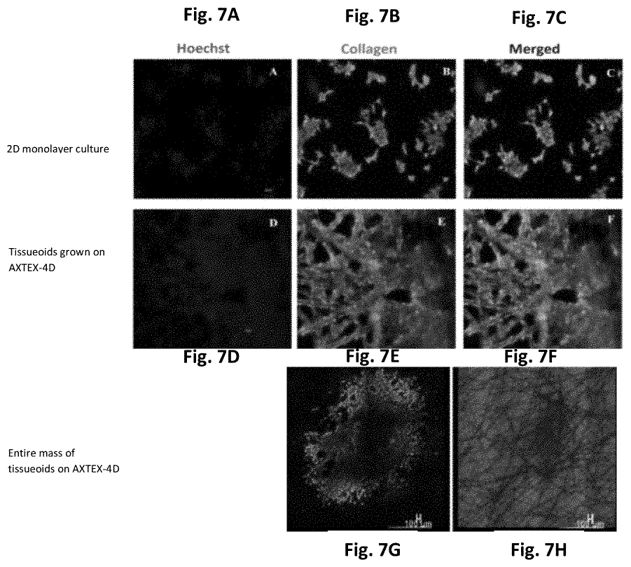

Cell-extra cellular matrix interaction plays an important role for the tumor growth and invasion and serves as a crucial component of tumor microenvironment. Presence of collagen as ECM component involves in cancer fibrosis. Collagen in presence of other components like hyaluronic acid, fibronectin, laminin and matrix metalloprotease influences cancer cell activity. Tissuoids generated from MCF-7 cell line, grown on AXTEX-4D system, were observed to produce collagen (FIG. 7). ECM formation was more contiguous in case of tissueoids grown on AXTEX-4D as compared to the 2D monolayer cultures.

### Example 6: Analysis of 2D and 3D Cell Culture Sensitivity to Drugs

The MCF-7 cell line was grown as 2D monolayer cultures as well as tissueoids grown on 96 well plate, where the spheroids were cultured on top of the membrane and incubated for 1 to 3 days. A cell number of approximately 5×10³ cells per spheroid were added to each well in 96 well plate either pre-coated with 1.5% agarose in tissue culture coated 96 well plate or without agarose in tissue culture uncoated 96 well plate. After attachment of spheroids, media was replaced with fresh media in presence and absence of drugs. In 2D culture 5×10³ cells were seeded in each well of a 96 well plate. Drug treatment was initiated after attachment of cells for 48-72 hrs.

As shown in FIG. 10, MCF-7 cells grown in 2D culture or on AXTEX-4D base matrix system, were treated with doxorubicin (1-5 μM), the cells growing in 3D format showed greater resistance to growth arrest or killing compared to the cells cultured as 2D monolayer (FIG. 10). FIG. 10A-FIG. 10B shows effect of doxorubicin in different concentrations (1-5 μM) on cell viability as they are grown in a 2D monolayer, and FIG. 10C shows effect of doxorubicin in different concentration (1-5 μM) on cell viability as grown as a 3D tissueoid on AXTEX-4D base matrix system. As shown in FIG. 11A, cells and tissueoids were treated with different doses (1-5 μM) of doxorubicin and partial resistance was observed in tissueoids with 1 μM doxorubicin compared to vehicle control.

FIG. 11B shows combined effect of both doxorubicin and bevacizumab on growth of tissueoids of MCF-7. The MCF-7 tissueoids, grown on AXTEX-4D base matrix system, were initially serum starved for 5 hrs and treated with either 100 ng/ml VEGF-165 (Vascular endothelial growth factor-165, which is a splice variant or isoform, Cat No. 293-VE-010, R&D systems) alone or in presence of 1 μM doxorubicin and 25 μg/ml of bevacizumab (sourced from Roche, 100 mg/4 ml) for 6 days at 37° C. and 8% C0₂. Viability was assessed by using prestoblue. The drug susceptibility was analysed by fluorescence based studies (Excitation 485 nm/Emission 595 nm) using prestoblue.

Tissueoids grown on AXTEX-4D base matrix system showed greater efficacy (about 57%) to prevent cell growth even at 1 μM doxorubicin in presence of bevacizumab at 25 μg/ml.

### Example 7. Evaluation of the Tissueoids Grown on AXTEX-4D Base Matrix System by Fluorescence Microscopy

As a process for conducting immunofluorescence analysis, samples were fixed with 4% PFA for 15 mins and was washed with PBS for 3 times, 5 mins each. Samples were permeabilized with 0.1% triton-x. Staining was performed with anti-collagen type-I antibody (green) in 1:50 dilution, DAPI (blue-nucleus stain) on Day 7 and observed under fluorescence microscopy. The ECM formation in the tissueoids formed on AXTEX-4D system was observed to be more contiguous as seen in images (FIG. 7A-FIG. 7F) as compared to that of cells cultured in 2D monolayer. (Magnification 10×). The samples were analyzed using ApoTome microscope.

### Example 8. Confocal Analysis

Growth of tissueoids in 3D was visualized using confocal microscopy. For performing confocal analysis, the samples were fixed, stained and analysed using Leica TCS SP8. In this example, spheroids of MCF-7 cells were prepared (as described herein) and added on the top of the membrane, and incubated to obtain growth of tissueoids.

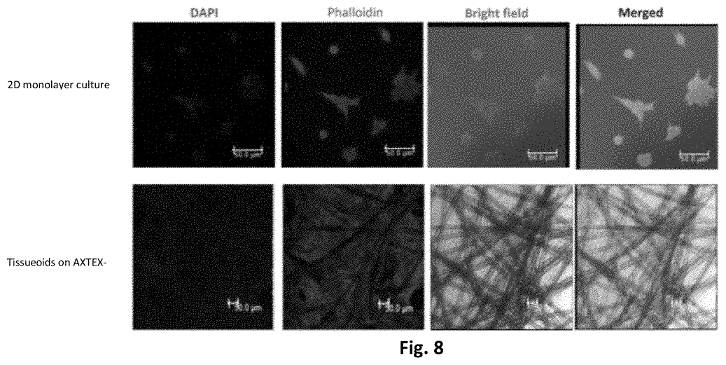

Cells were stained for F-actin using phalloidin and for nuclear staining using Hoechst dye. Cells were fixed with the fixatives and blocked for 30 mins at RT in PBS with 1% BSA. Afterwards, the specimens were washed in PBS, stained for actin followed by counter-staining with Hoechst for nucleus visualisation. Phalloidin staining was done using 1:1000 dilution for 40 mins at 25° C. Nuclear staining was performed using Hoechst at 1:1000 dilution in PBS for 15 minutes at 25° C. (FIG. 8). Photographs were taken at 10× magnification for 3D and 40× magnification for 2D using Leica confocal microscopy (Leica SP8).

To analyse the expression of collagen in tissueoids, cells were fixed and blocked as described earlier. Tissueoids of MCF-7 cell lines were stained with anti-collagen I antibody in 1:50 dilution for 16 hours. Nucleus staining was performed using DAPI. Photographs were taken at same magnification described before.

To analyse the proliferation of longevity of tissueoids derived from PC3 cell line, tissueoids were stained with calcein AM for 30 minutes as per manufacturer's protocol. As shown in FIG. 6, tissueoids generated from PC3 cell line grown on base matrix AXTEX-4D are viable and able to proliferate up to 250 days.

To analyse proliferation and viability of PC3 tissueoids, calcein AM (Thermo Fisher) staining was performed at different time points (day 3, day 25, day 108 and day 250). PC3 tissueoids were stained with 1 μM of calcein AM for 30 minutes and kept at 37° C. and 8% CO₂. Then tissueoids were analysed by confocal microscopy, the results showing increase in cell number viability as shown by Calcein AM staining (FIG. 6)

Confocal analysis data showed tissue like organization of tissueoids of the MCF-7 cell line with contiguity of the cells clearly visible. This is in contrast to the picture seen from the cells grown in Petri dish as a 2D format in which the cells have defined edges and margins and are non-contiguous (FIG. 8).

### Example 9: Small Cell Numbers of Initial Sample as Inocula for Growth of Tissueoid on AXTEX-4D

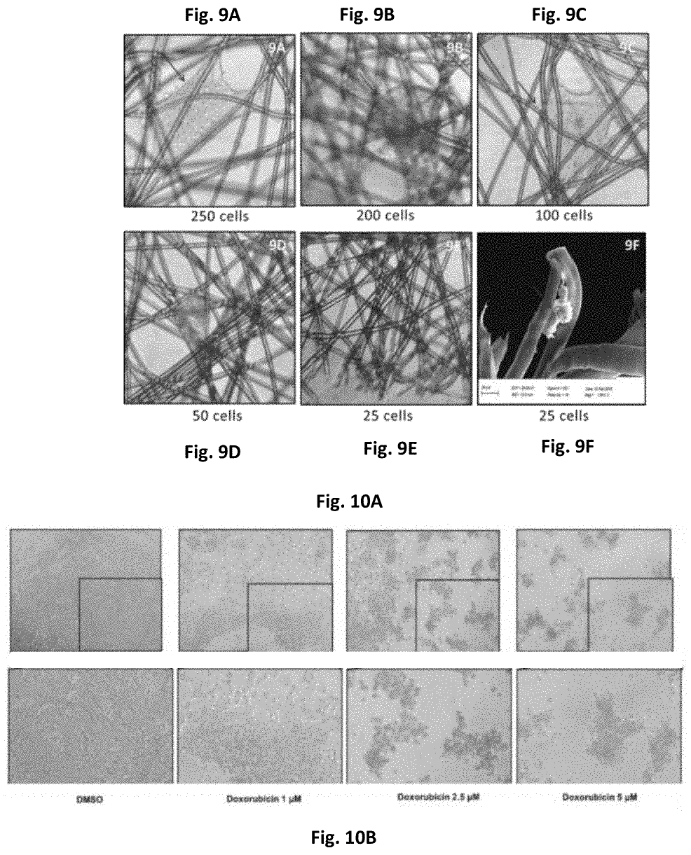

Data showed that as few as 25 cells successfully grew and formed a tissueoid on the base matrix system. Analysis was done by making cell suspension by dilution method such that 20 μl of the media contained a precise number of cells, ranging from 25 to 250 cells. The drop was pipetted onto the inner surface of a lid filled with PBS at the bottom. After 24 hrs, the inner lid was inverted and the drops were re-suspended in a fresh media. Spheroids were analyzed by phase contrast microscopy and were added on the top of base matrix placed in a tissue culture plate (24 or 96 well plates) and incubated in a humidified incubator at 37° C. and 8% CO₂. The attachment and growth of the tissueoids using different inoculating numbers of cells was investigated under phase contrast and scanning electron microscope (FIG. 9).

As illustrated in the figure, different cell number of MCF-7 cell line were used as starting material to grow spheroids and 3D tissueoids, ranging from about 250 cells to as few as less than 25 cells. Spheroids were grown on the 3D base matrix system as shown in Figs. FIG. 9A, FIG. 9B, FIG. 9C, FIG. 9D, and FIG. 9E using phase contrast microscope (10× magnification) and FIG. 9F using SEM analysis (1500× magnification). The photographs show growth of tissueoids and clearly showed that as few as 25 cells were needed to create a tissueoid on the base matrix system provided herein.

### Example 10: Viability Time Course of Cell Cultures on Base Matrix System and Culture Duration

Growth and viability of tissueoids grown on base matrix, AXTEX-4D was observed to have extended to more than 1 year (approximately 364 days, FIG. 13, lower panel) for PC3 and approximately 3 months for HepG2 (-82 days, FIG. 13, upper panel) and close to 130 days for MCF-7 tissueoids (We have analysed and observed this phenomenon with three different cell lines (HepG2, MCF-7 and PC3) on the AXTEX-4D base matrix system base matrix system (19 gm/m²).

Further, viability of PC3 tissueoid was analysed at day 100 of its growth by FACS analysis using LIVE/DEAD stain and it was found that out of gated population i.e. (˜75%), 47. % cells are live and 18.68% cells are dead suggesting almost 60% viability of PC3 tissueoids even after 100 days of culture .

Distinct advantages of prolonged growth and viability is provided by data herein showing ability to mimic tissue like conditions ex vivo for a longer period so that different assays are performed for an extended period of time, as a method to obtain the drug sensitivity data to design the best therapeutic regimen for a patient.

### Example 11

### A Plurality Of Different Cell Lines were Grown on the Base Matrix System:

The tissueoid generation methods and systems described in this application are a universal base matrix system that was shown in examples herein capable of use for culture of different types of cells in a 3D format. The following cell lines were successfully grown on the fabric base matrix systems using the process described above in Example 2 (FIG. 1B, FIG. 1C, FIG. 2, FIG. 3A, FIG. 4A-FIG. 4D,) Cancerous cell lines: MCF-7: breast cancer cell line; adenocarcinoma; HepG2: liver carcinoma; epithelial cells; PC3: prostate cancer cell line; adenocarcinoma; A375: skin melanoma; epithelial cell line; HT-29: colorectal; adenocarcinoma and non-malignant cell line CHO-K1 cells (stably expressing surface protein); HEK-293; NIH3T3 Fibroblasts. This system has also been tested for growth of tissueoids derived from primary tumor tissues like colon, gastric, lung and thyroid (represented in FIG. 3B and, FIG. 5).

### Example 12: Co-Culture and Tri-Culture of Different Cell Mixtures on the Base Matrix System

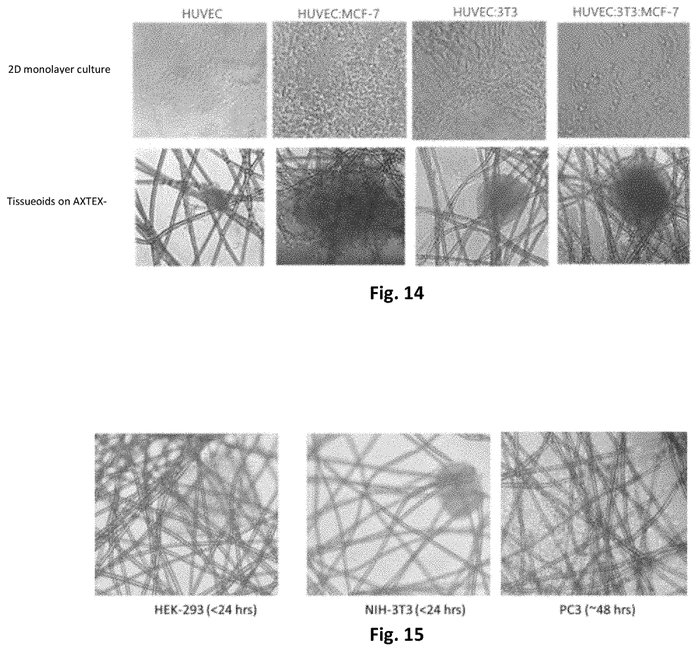

Tissueoids were generated from mixed cell populations by co-culturing two or more cell lines. Co-culture of each of the combinations were analysed by taking either breast cancer cell line (MCF-7) and endothelial cells (HUVEC) or endothelial cells (HUVEC) and fibroblast (NIH-3T3) in 1:1 ratio respectively. These were grown in 2D monolayer format and on the AXTEX-4D base matrix system, as shown in FIG. 14. The tissueoids were seen to grow very efficiently.

Tissueoids were generated of mixed cell populations by co-culturing three cell lines. The cell suspension of MCF-7 cell line was mixed with NIH-3T3 and HUVEC cell line in 1:2:1 ratios. These mixed cell populations were grown on the AXTEX-4D and the data is shown in FIG. 14, indicating that the populations grew very efficiently.

Producing and analyzing co-cultures and tri-cultures using the tissueoid base matrix system was envisioned as useful in studying cell-cell interaction, drug discovery and development and also for patient treatment regimen, especially for the immune-oncology and infectious disease base matrix systems FIG. 14 .

### Example 13: Primary Cells and Tissue Samples from Patients Grown on the AXTEX-4D Base Matrix System

Primary cell lines and samples from tissue biopsies from oncology patients were grown on the base matrix system as tissueoids. Tumor tissue specimens were taken as suspension culture or as explant and grown on AXTEX-4D base matrix system. The growth of tumor tissue on the base matrix system is shown as an example in FIG. 3B and FIG. 5 demonstrating that AXTEX-4D base matrix system can be effectively and universally used to generate tissueoids from primary tissue samples/biopsies.

### Example 14: Reduction in Time to Grow the Tissueoid on the Base Matrix System and Initiate the Assays

Observations herein report a time interval equal to or less than 24 hrs for the cells from cell lines/primary cells to attach to the AXTEX-4D base matrix system and initiate growing as tissueoids. The tissueoid was observed to be suitable for analyses for drug sensitivity and resistance appropriate to therapeutic drug regimen assays. In certain cell lines, it was observed that the cells required somewhat more time, but generally for human cell lines no more time than 72 hours was required for the attachment on the base matrix system and to start growth as a tissueoid. This rapidity of culture of tissueoids addresses a long felt need and the key critical factor for any patient-drug related studies that is factor of time and makes it a four-dimensional system. (FIG. 15).

### Example 15: Cell Factory

AXTEX-4D base matrix system sustains the growth of tissueoid for longer time duration. It is envisioned that for large-scale production of cells, vaccines, and therapeutic proteins, antibodies, secretory proteins the 3D systems and methods and format provided herein are very useful. The system is convenient to handle, requires no special tubing and increased antibody production was observed as a function of time as is shown in FIG. 16.

CHO-DG44 cells stably expressing tocilizumab, an antilL-6R antibody, growing on AXTEX-4D base matrix system allowed increased number of cells in a more compact space with increased longevity and better productivity of the antibody expressing cells (FIG. 16). This confirms use of AXTEX-4D base matrix system for cell factory for biotherapeutic production of biologics and vaccines.

### Example 16: Angiogenesis Base Matrix System

Endothelial cell dysfunction has a role in diabetes, pulmonary diseases, inflammatory diseases, cardiovascular diseases and immune diseases etc. Angiogenesis is a critical process for tissue development, wound healing and tumor progression. The methods utilising 3D format provided useful insights for studying angiogenesis or tumor microenvironment screening for inhibitors of anti-angiogenic drug.

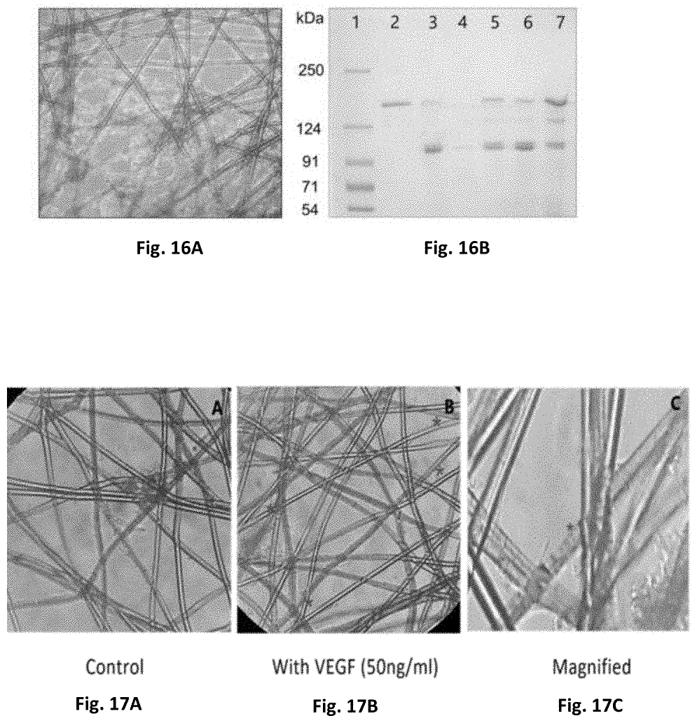

Tissueoids generated from loosely compact HUVEC cells were grown on AXTEX-4D system in presence of VEGF-165, which is a potent mediator of angiogenesis. FIG. 17 represents growth of tissueoids along with tube like structure formation after treatment with 50 ng/ml of VEGF treatment for 72 hrs.

The 3D methods and systems provided herein have yielded important insights into angiogenesis and creation of the tumor microenvironment and the need for screening potential anti-angiogenesis drugs in a system that closely resembles that of a tumor in vivo.

1. A device for growth of cells comprising at least one sterile culture chamber, each chamber containing a sterile non-woven fabric base matrix system for receiving and supporting an inoculum selected from the group of: a spheroid from a hanging drop culture, a volume of a cell culture, and a primary culture of a biopsy, and a biopsy explant, each chamber having a bottom and sides for holding culture medium, the base matrix system and cells, for growth of the cells in three dimensions (3D).

2. The device according to claim 1, the fabric of the base matrix system comprising a non-woven matrix of polymer fibers consisting of at least one selected from the group of: PET, PP, PBT, glass fiber, and cotton.

3. The device according to claim 1, the fabric of the base matrix system having a density of approximately 10-50 gm/m² and a thickness of at least about 0.05 mm and less than about 5 mm.

4. The device according to claim 1, further comprising the inoculum or a spheroid or a tissueoid cultured in the chamber from the inoculum.

5. The device according to claim 4, wherein the cells are mammalian or avian in origin.

6. (canceled)

7. The device according to claim 4, wherein the cells are human.

8. The device according to claim 4, wherein the cells following growth in the chamber maintain cellular architecture, the architecture comprising at least one of an intracellular structure and an extracellular structure selected from: a component of an extracellular matrix, for example collagen or vascular tubules; and an intracellular structure, for example, collagen or vascular tubules.

9-13. (canceled)

14. A method of making a device for three dimensional growth of cell tissueoids comprising:
providing samples of cells selected for inoculation on the device from a biopsy of a patient, an explant from biopsy, a cell culture in a tissue culture plate, and/or hanging drop cultured cell spheroids to obtain a resulting plurality of multicellular inocula;
transferring at least one of the inocula to a corresponding at least one of culture vessels each containing a non-woven fabric base matrix system and growth media; and,
incubating the vessels to obtain the three dimensional tissueoids of cells in the device.

15. The method according to claim 14, wherein providing the inoculation comprises preparing each sample of cells to contain less than about 1,000 cells, less than about 500 cells, less than about 250 cells, less than about 100 cells, or less than about 25 cells.

16. A method of use for analysis of cell-drug sensitivity of a device for three dimensional growth of tissueoids on a non-woven fabric support base matrix system comprising:
contacting at least one test chamber of tissueoids cultured from cells or a tissue from a patient biopsy, with at least one concentration of a drug; and,
comparing growth and viability of the cells in the tissueoids with a control chamber with absent drug but otherwise identical.

17. The method of claim 16, wherein the at least one concentration is a plurality of concentrations of the drug in a corresponding plurality of test chambers;
and/or, wherein the drug is a combination of at least two drugs in at least two test chambers.

18. The method of claim 16, wherein the test chamber and the control chamber contain tissueoids cultured from tumor biopsy from the patient.

19. The method of claim 16, wherein an additional control chamber contains a tissueoid comprising non-tumor normal cells from the patient.

20. The method according to claim 19, wherein the drug is an anti-cancer chemical agent or an anti-cancer antibody or binding protein or a peptide.

21. The method according to claim 16, wherein a plurality of test chambers contain a combination of two or more drugs at two or more concentrations.

22. The method according to claim 20, wherein in addition to the anti-cancer agent a second drug is selected from: anti-bacterial, anti-inflammatory, anti-viral, anti-helminthic, anti-angiogenesis, and anti-psychotic.

23. A device for growth and drug sensitivity characterization of cells from a subject with cancer, the device comprising a plurality of sterile culture chambers, each chamber containing a sterile non-woven polyethylene terephthalate (PET) fabric base matrix system for receiving and supporting an inoculum of subject cells selected from the group of: a spheroid from a hanging drop culture, a volume of a cell culture, and a primary culture of a biopsy, wherein a test plurality of cultures originates from cancerous tissue from the subject, and a control culture or biopsy originates from normal tissue from the subject, each chamber having a bottom and sides for holding culture medium, the base matrix system, and cells, for characterization of growth and viability of the cells in three dimensions (3D) under a set of variable medium constituents.

24. (canceled)

25. The device according to claim 23, wherein the sterile culture chambers are wells in a multi-well culture dish, for example, a 24 well culture dish or a 96 well culture dish.

26. (canceled)

27. The method according to claim 14, further comprising generating the tissueoids from the inocula in less than 72 hours, less than 48 hours, or less than 24 hours.

28. A cell culture and artificial tissue production device comprising at least one sterile culture chamber, each chamber containing cells and a sterile non-woven polyethylene terephthalate (PET) fabric base matrix system for receiving and supporting an inoculum of cells selected from the group of: spheroids created on a matrix or from a hanging drop culture, volumes of a cell culture, biopsy explant and primary cultures of a biopsy, each chamber having a bottom and sides for holding culture media, the base matrix systems, and cells, each chamber having an inlet port for addition of fresh culture medium and an outlet port to drain spent medium.

29-37. (canceled)
